(19) United States
(12) Patent Application Publication
(10) Pub. No.: US 2007/0135751 A1
(43) Pub. Date: Jun. 14, 2007

(54) Medical devices

(76) Inventors: Paul D. DiCarlo, Middleboro, MA (US); Raymond Lareau, Westford, MA (US); Barbara Bell, Sudbury, MA (US); Christopher J. Elliott, Hopkinton, MA (US); Pavel Mikhailovich Karavaev, Saint-Petersburg (RU); Leonid Malinin, Newton, MA (US); Alexander Vladimirovich Kudryavtsev, Moscow (RU); Valentin Vassilievich Titov, Moscow (RU); Victor Efimovich Minaker, Moscow (RU); Marina Mikhailovna Ksenofontova, Moscow (RU); Eugene Dmitrievich Bush, Moscow (RU); Alexey Mikhailovich Blinov, Ramenskoe (RU); Leonid Yurevich Pustov, Moscow (RU)

Correspondence Address: FISH & RICHARDSON PC, P.O. BOX 1022, MINNEAPOLIS, MN, 55440-1022, US

(21) Appl. No.: 11/297,971

(22) Filed: Dec. 9, 2005

(51) Int. Cl. A61N 1/30

(52) U.S. Cl. 604/19

(57) ABSTRACT

Medical devices, such as catheters, ports, and stents, are disclosed.

### TECHNICAL FIELD

The invention relates to medical devices, such as, for example, catheters.

### BACKGROUND

Agents, such as therapeutic agents, can be delivered systemically, for example, by injection through the vascular system or oral ingestion, or they can be applied directly to a site where treatment is desired. In some cases, a catheter can be used to deliver a therapeutic agent to a target site.

### SUMMARY

In one aspect, the invention features an access catheter including a generally tubular member insertable through the skin into a body of a subject and including a proximal portion positionable proximate to the skin of the subject and a distal portion. The generally tubular member includes a tubular section including a polymer and at least about three percent by volume silver, and exhibits radiopacity and antimicrobial activity.

In another aspect, the invention features an implantable medical device (e.g., a catheter, a port, a stent) including a composite including a polymer and at least about three percent by volume silver. The implantable medical device exhibits radiopacity and antimicrobial activity.

In an additional aspect, the invention features an implantable medical device (e.g., a catheter, a port, a stent) including a plurality of spaced portions including silver. At least one of the portions includes at least three percent by volume silver.

In a further aspect, the invention features a urethral stent including a generally tubular member including at least about three percent by volume silver and exhibiting radiopacity and antimicrobial activity.

In an additional aspect, the invention features an implantable medical device (e.g., a catheter, a port, a stent) including a body including at least about three percent by volume silver and a polymer, a metal, a metal alloy, and/or glass. The medical device (e.g., the body of the medical device) exhibits radiopacity and antimicrobial activity.

In another aspect, the invention features a method including delivering an implantable medical device (e.g., a catheter, a port, a stent) including a section including a polymer and at least about three percent by volume silver into a body of a subject, and viewing the section using X-ray fluoroscopy and/or ultrasound. The section exhibits antimicrobial activity.

Embodiments can also include one or more of the following.

The tubular section can be selectively located in the proximal portion of the generally tubular member. The access catheter can include a single tubular section located only in the proximal portion of the generally tubular member. The access catheter can include a plurality of spaced tubular sections including a polymer and at least about three percent by volume silver. The tubular section can include a layer including the polymer and/or a layer including the silver. The layer including the silver can be supported by the layer including the polymer. The layer including the polymer can be supported by the layer including the silver. The tubular section can include two layers that each include silver. The layer including the polymer can be disposed between the two layers that each include silver. The silver and the polymer can be in the form of a composite. The silver can be in the form of a coating on the polymer. The coating can be on an interior surface of the tubular section. The tubular section can include silver and the polymer in the form of a composite and can also include the silver in the form of a coating. The tubular section can include at most about 60 percent by volume silver (e.g., at most about 40 percent by volume silver, at most about 20 percent by volume silver, at most about 15 percent by volume silver, at most about 10 percent by volume silver), and/or at least about four percent by volume silver (e.g., at least about five percent by volume silver). The tubular section can include more than five percent by volume silver. The tubular section can include at least about 0.5 percent by weight silver (e.g., at least about five percent by weight silver, at least about 10 percent by weight silver, at least about 20 percent by weight silver, at least about 50 percent by weight silver) and/or at most about 70 percent by weight silver (e.g., at most about 50 percent by weight silver, at most about 20 percent by weight silver, at most about 10 percent by weight silver, at most about five percent by weight silver).

The catheter can include a second tubular section that can include a polymer and at least about three percent silver. The second tubular section can be located in the distal portion of the generally tubular member and can be spaced from the tubular section in the proximal portion of the generally tubular member.

The silver can include elemental silver. The silver can be in the form of a silver complex, such as a silver salt. The silver can be in the form of particles. The particles can have a maximum dimension of at most about 100 microns (e.g., at most about 50 microns, at most about 25 microns, at most about 10 microns, at most about five microns, at most about one micron, at most about 500 nanometers, at most about 250 nanometers, at most about 100 nanometers).

The generally tubular member (e.g., the tubular section of the generally tubular member) can include at least one radiopaque material selected from barium sulfate, bismuth trioxide, gold, platinum, bismuth oxychloride, bismuth subcarbonate, iridium, tungsten, and combinations thereof.

The catheter can be an access catheter. The catheter can include a generally tubular member including a layer including the composite. The generally tubular member can include another layer including silver.

The implantable medical device can exhibit radiopacity and antimicrobial activity.

Embodiments can include one or more of the following advantages.

In embodiments, an implantable medical device is provided that includes silver in a selected amount, form, and location such that the medical device exhibits both radiopacity and antimicrobial activity, while also exhibiting sufficient or improved mechanical properties, such as flexibility and strength, to enhance the therapeutic function. For example, an access catheter is provided with silver at a location proximate to the skin, a region prone to high infection risk, in an amount to enable fluoroscopic observation while enhancing pushability and kink resistance of the catheter in its proximal portions and maintaining sufficient flexibility in its more distal portions to permit the catheter to be threaded through a tortuous vasculature. The silver can be provided as small particles compounded in a composite which is formed into the catheter body, a coating over the catheter body, or both. The silver may be provided only in proximal locations of the catheter body or in select proximal and distal locations (such as the proximal and distal end regions) or intermittently along the length of the catheter body or along the entire catheter body.

In some embodiments, the location of a medical device (e.g., a catheter) including silver can be readily ascertained (e.g., using X-ray fluoroscopy). In certain embodiments, the medical device may not include any other radiopaque materials, but may still be visible under X-ray fluoroscopy. In some embodiments in which silver is included in a medical device, the silver can render the medical device radiopaque without adversely affecting the properties of the medical device. As an example, in certain embodiments, a medical device formed of a composite including a polymer and silver particles can be viewed using X-ray fluoroscopy, and can also have mechanical properties (e.g., strength) that are comparable to those of a medical device that is formed of the same polymer, but that does not include silver particles. In some embodiments, the presence of silver particles in a composite in a medical device can have little or no effect on a polymer in the composite. As an example, the silver particles may be present in a relatively small percent by volume, and may have little or no effect on the properties of a polymer in the composite (e.g., so that the properties of the overall composite are similar to, or the same as, the properties of the virgin polymer). For example, the silver particles may not have a substantial effect on the interactions between polymer chains in the composite, and/or on the chemical stability of the polymer in the composite. In certain embodiments, the composite may exhibit chemical resistance, such as a resistance to alcohol. A resistance to alcohol can, for example, limit the likelihood of the composite absorbing alcohol and swelling (e.g., during routine cleaning of the medical device).

In certain embodiments, a medical device (e.g., a catheter) that includes silver but does not include any radiopaque structures (e.g., radiopaque markers) can be viewed using X-ray fluoroscopy. A medical device that does not include radiopaque structures may, for example, be relatively easy and/or inexpensive to manufacture.

In certain embodiments, a medical device (e.g., a catheter) that includes silver can exhibit antimicrobial activity. A medical device that exhibits antimicrobial activity may, for example, be relatively unlikely to result in an infection during use. In some embodiments, a medical device that includes silver can be used to treat a subject for a relatively long period of time (e.g., at least about one month) without having an adverse effect on the subject (e.g., without resulting in an infection in the subject).

In certain embodiments, a medical device (e.g., a catheter) including silver can be viewed using X-ray fluoroscopy, and can also exhibit antimicrobial activity.

In some embodiments, the presence of silver in a medical device can enhance ultrasound imaging of the medical device. For example, in some embodiments, the addition of silver particles to a catheter can result in variations in the density of the catheter, and/or can result in irregularities on the surface of the catheter. This density variability and/or these irregularities can enhance imaging of the catheter using ultrasound (e.g., by changing the extent of sound absorption by the catheter). In certain embodiments in which a medical device includes silver particles of varying sizes, the size variability of the silver particles can enhance the ultrasound visibility of the medical device.

In some embodiments, a medical device (e.g., a catheter) including silver can be viewed using ultrasound, and can also exhibit antimicrobial activity.

In certain embodiments, a medical device (e.g., a catheter) can be viewed using X-ray fluoroscopy and using ultrasound, and can also exhibit antimicrobial activity.

In some embodiments, a medical device (e.g., a catheter) including silver can have a relatively low profile. For example, in certain embodiments, a catheter including a composite including a polymer and silver particles dispersed within the polymer can have a relatively low profile. In some embodiments, a silver coating (e.g., a coating formed of silver particles) can be added to a medical device without substantially increasing the profile of the medical device.

In certain embodiments, silver particles may be unlikely to become dislodged and/or detached from a medical device (e.g., a catheter) including the silver particles (e.g., upon delivery of the medical device to a target site). For example, in some embodiments in which a catheter includes a composite including a polymer and silver particles dispersed within the polymer, the polymer can help to retain the silver particles.

In some embodiments, a medical device (e.g., a catheter) that includes silver can exhibit enhanced mechanical properties. For example, in certain embodiments, a catheter that includes a composite including a polymer and silver particles may be relatively strong and/or stiff. In some embodiments, a relatively strong and/or stiff catheter may be unlikely to kink and/or buckle during use (e.g., during delivery to a target site).

In some embodiments, a composite including a polymer and silver (e.g., silver particles) can be relatively biocompatible. In certain embodiments, a medical device (e.g., a catheter) including the composite (e.g., in the form of a coating on the medical device) can be relatively thromboresistant and/or can have enhanced fatigue strength. In some embodiments, a coating formed of the composite can have a relatively low tackiness, can experience relatively little friction upon contacting other surfaces, and/or can be relatively wear-resistant. In certain embodiments, a medical device including the composite (e.g., in the form of a coating on the medical device) can be delivered to a target site without using lubricants.

Features and advantages are in the description, drawings, and claims.

### DESCRIPTION OF DRAWINGS

FIG. 1A is a partial cross-sectional view of a body of a subject after a portion of an embodiment of a catheter has been delivered into the vasculature of the subject.

FIG. 1B is a partial cross-sectional view of the body of FIG. 1A, illustrating the position of the catheter of FIG. 1A on and within the body of the subject.

FIG. 1C is a side view of the catheter of FIGS. 1A and 1B.

FIG. 1D is a cross-sectional view of the catheter of FIG. 1C, taken along line 1D-1D.

FIG. 1E is a side view of the catheter of FIGS. 1A-1D.

FIG. 2A is a side view of an embodiment of a catheter.

FIG. 2B is a cross-sectional view of the catheter of FIG. 2A, taken along line 2B-2B.

FIG. 3A is a side view of an embodiment of a catheter.

FIG. 3B is a cross-sectional view of the catheter of FIG. 3A, taken along line 3B-3B.

FIG. 4A is a side view of an embodiment of a catheter.

FIG. 4B is a cross-sectional view of the catheter of FIG. 4A, taken along line 4B-4B.

FIG. 5A is a side view of an embodiment of a catheter.

FIG. 5B is a cross-sectional view of the catheter of FIG. 5A, taken along line 5B-5B.

FIG. 6A is a side view of an embodiment of a catheter.

FIG. 6B is a cross-sectional view of the catheter of FIG. 6A, taken along line 6B-6B.

FIG. 6C is a cross-sectional view of the catheter of FIG. 6A, taken along line 6C-6C.

FIG. 7A is a side view of an embodiment of a catheter.

FIG. 7B is a cross-sectional view of the catheter of FIG. 7A, taken along line 7B-7B.

FIG. 8A is an illustration of the placement of an embodiment of a catheter in a vessel of a subject.

FIG. 8B is a side view of the catheter of FIG. 8A.

FIG. 8C is a cross-sectional view of the catheter of FIG. 8B, taken along line 8C-8C.

FIG. 9A is a side view of an embodiment of a catheter.

FIG. 9B is a cross-sectional view of a component of the catheter of FIG. 9A, taken along line 9B-9B.

FIG. 10A is a side perspective view of an embodiment of a port system.

FIG. 10B is a cross-sectional view of the port system of FIG. 10A, taken along line 10B-10B.

FIG. 10C is an illustration of the placement of the port system of FIGS. 10A and 10B in a body of a subject.

FIG. 10D is an illustration of the injection of a therapeutic agent into the port system of FIGS. 10A and 10B.

FIG. 11 is a side view of an embodiment of a catheter component.

FIG. 12 is a side view of an embodiment of a catheter component.

### DETAILED DESCRIPTION

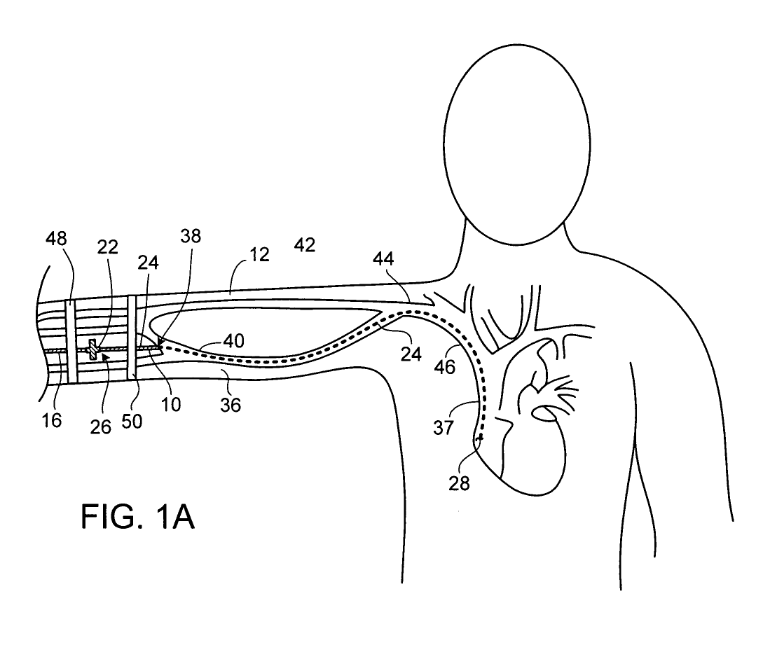

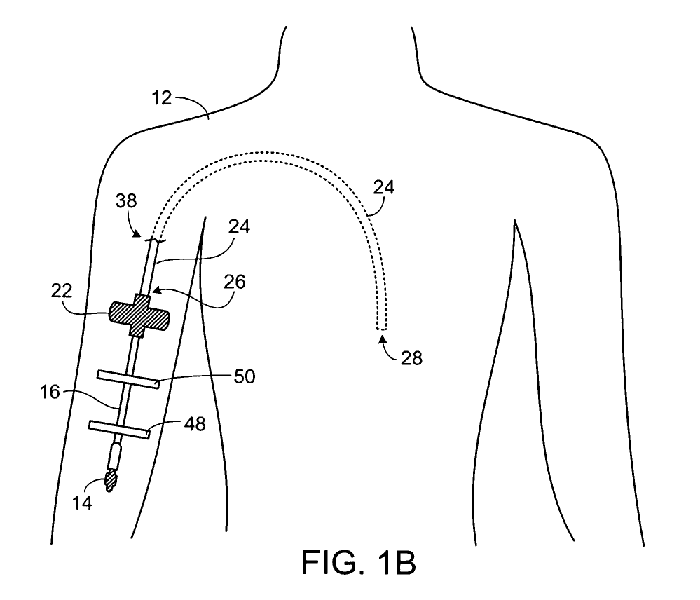

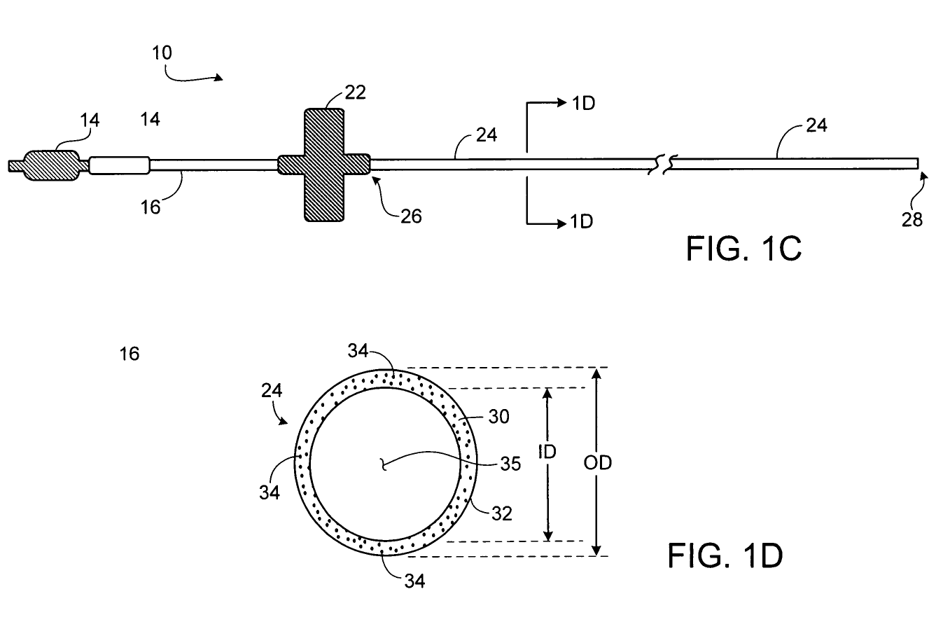

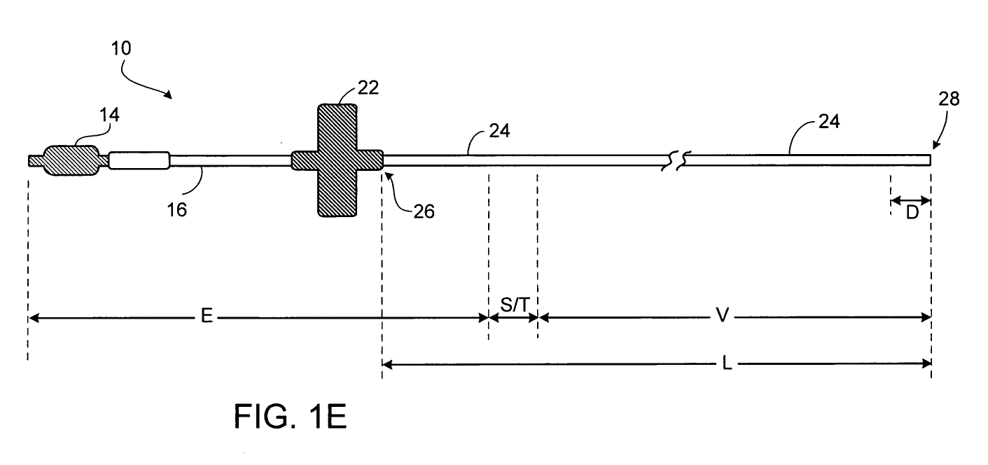

Referring to FIGS. 1A and 1B, an access catheter 10 extends from a region outside of the body 12 of a subject through tortuous vasculature within the body to a location, such as the heart, where a therapeutic agent (e.g., a drug) can be delivered. Access catheter 10 exhibits sufficient flexibility and other mechanical properties so that it can be threaded along and maintained within the vasculature. Access catheter 10 also includes silver, which makes access catheter 10 radiopaque so that it can be monitored by X-ray fluoroscopy, and provides access catheter 10 with antimicrobial activity. In some embodiments, access catheter 10 can be used to deliver therapeutic agents into body 12 over a relatively long period of time (e.g., from about one week to about 30 weeks). In certain embodiments, access catheter 10 can be used for pain drug delivery and/or pain management, and/or can be used for Total Parenteral Nutrition (TPN) infusion.

Access catheter 10 can be used, for example, to deliver therapeutic agents into the superior vena cava 37 of body 12 via generally tubular member 24. As shown in FIG. 1A, a target vein (here, basilic vein 40) in right arm 36 of body 12 is located and accessed by inserting a needle (not shown) into a location 38 in right arm 36. Once the needle has accessed basilic vein 40, a guidewire (not shown) is threaded through the needle and into basilic vein 40. The guidewire is then threaded through basilic vein 40, axillary vein 42, subclavian vein 44, and brachiocephalic vein 46, until it reaches superior vena cava 37. After the guidewire has been positioned, the needle is removed and an introducer sheath (not shown) is threaded over the guidewire until the introducer sheath reaches superior vena cava 37. Then, the guidewire is removed, and generally tubular member 24 of access catheter 10 is advanced through the introducer sheath until distal end 28 of generally tubular member 24 reaches superior vena cava 37. During delivery of generally tubular member 24 into superior vena cava 37, the location of generally tubular member 24 can be ascertained using X-ray fluoroscopy. After generally tubular member 24 has been placed, the introducer sheath is proximally withdrawn over generally tubular member 24. The position of access catheter 10 is then secured using adhesive strips 48 and 50. Thereafter, one or more therapeutic agents can be flowed through a lumen 35 (FIG. 1D) of generally tubular member 24 (e.g., by injecting the therapeutic agents into valve 14) and into superior vena cava 37.

FIG. 1C provides an enlarged view of access catheter 10. As shown in FIG. 1C, access catheter 10 includes a valve 14 that is connected to a line 16, which in turn is connected to a hub 22. Hub 22 is in fluid communication with a generally tubular member 24 having a proximal end 26 and a distal end 28. Referring also now to FIG. 1D, generally tubular member 24 includes lumen 35, and is formed of a composite 30 including a polymer 32 and silver particles 34 (formed of elemental silver) dispersed within polymer 32. As shown in FIG. 1D, generally tubular member 24 has an inner diameter ID and an outer diameter OD.

The mechanical properties, radiopacity, and antimicrobial activity of access catheter 10 are selected by controlling the amount, location, and form of the silver in access catheter 10. The amount of silver particles 34 dispersed within polymer 32 of composite 30 can be selected to provide radiopacity to one or more portions (e.g., all) of generally tubular member 24, without also making those portions of generally tubular member 24 too dark to be viewed adequately under X-ray fluoroscopy. The relative radiopacity of a material (e.g., composite 30) can be measured using, for example, ASTM F640-79(2000) (Test Method B). In some embodiments, at least a portion (e.g., all) of generally tubular member 24 and/or composite 30 can include at least about three percent by volume (e.g., at least about four percent by volume, at least about five percent by volume, at least about six percent by volume, at least about seven percent by volume, at least about eight percent by volume, at least about nine percent by volume, at least about 10 percent by volume, at least about 15 percent by volume, at least about 20 percent by volume, at least about 25 percent by volume, at least about 30 percent by volume, at least about 40 percent by volume, at least about 50 percent by volume), and/or at most about 60 percent by volume (e.g., at most about 50 percent by volume, at most about 40 percent by volume, at most about 30 percent by volume, at most about 25 percent by volume, at most about 20 percent by volume, at most about 15 percent by volume, at most about 10 percent by volume, at most about nine percent by volume, at most about eight percent by volume, at most about seven percent by volume, at most about six percent by volume, at most about five percent by volume, at most about four percent by volume), of silver particles 34. For example, in certain embodiments, at least a portion of generally tubular member 24 and/or composite 30 can include from about three percent by volume to about 15 percent by volume (e.g., from about five percent by volume to about 15 percent by volume, from about five percent by volume to about seven percent by volume) of silver particles 34. In certain embodiments, at least a portion of generally tubular member 24 and/or composite 30 can include more than five percent by volume (e.g., about 6.4 percent by volume) of silver particles 34.

In some embodiments, the percent by volume of silver particles 34 in composite 30 and/or in a portion of generally tubular member 24 formed of composite 30 can be measured prior to formation of composite 30. For example, the percent by volume of silver particles 34 in composite 30 can be measured as follows. First, prior to forming composite 30, the mass of silver particles 34 is measured using a balance, and the mass of polymer 32 is also measured (separately) using a balance. The mass of silver particles 34 is then divided by the density (mass per unit volume) of elemental silver to provide the volume of silver particles 34. Similarly, the mass of polymer 32 is divided by the density (mass per unit volume) of polymer 32 to provide the volume of polymer 32. The volume percent of silver in composite 30 is then calculated according to equation (1) below:

$$silver \% by volume = \frac{( volume of particles 34 )}{( volume of polymer 32 + volume of particles 34 )} \times 100 \% \qquad ( 1 )$$

In certain embodiments, the percent by volume of silver particles 34 in composite 30 and/or in a portion of generally tubular member 24 formed of composite 30 can be measured after formation of composite 30. For example, the percent by volume of silver particles 34 in a portion of generally tubular member 24 formed of composite 30 can be measured as follows. First, the portion of generally tubular member 24 is heated to melt polymer 32. Then, silver particles 34 in the portion are precipitated to separate polymer 32 from the silver particles. The masses of the polymer 32 and silver particles 34 in the portion are then measured as described above. Next, the volume of silver particles 34 in the portion is determined by dividing the mass of silver particles 34 by the density of elemental silver, and the volume of polymer 32 in the portion is determined by dividing the mass of polymer 32 by the density of polymer 32. The volumes of silver particles 34 and polymer 32 can then be used to determine the volume percent of silver in the portion of generally tubular member 24, as described above with respect to equation (1).

As described above, silver particles 34 are formed of elemental silver. The presence of silver particles 34 in composite 30 can result in composite 30 (and, therefore, generally tubular member 24) exhibiting enhanced antimicrobial activity. For example, silver particles 34 and/or oxidized silver particles 34 can limit or prevent the formation of biofilms on generally tubular member 24 by, for example, limiting or preventing germination and/or propagation of bacteria on generally tubular member 24. The presence of silver particles 34 in composite 30 can result in a reduced likelihood of infection of body 12 when generally tubular member 24 in implanted within body 12. The antimicrobial activity of generally tubular member 24 can be evaluated using, for example, ASTM E2149-01, a dynamic shake flask test for antimicrobial activity.

Composite 30 can include silver particles 34 of the same size, of different sizes, or some silver particles 34 of the same size and some of different sizes. In some embodiments, a silver particle 34 can have a maximum dimension (e.g., a diameter) of at most about 100 microns (e.g., at most about 75 microns, at most about 50 microns, at most about 25 microns, at most about 10 microns, at most about five microns, at most about one micron, at most about 500 nanometers, at most about 250 nanometers, at most about 100 nanometers, at most about 50 nanometers, at most about 25 nanometers, at most about 10 nanometers, at most about five nanometers, at most about two nanometers) and/or at least about one nanometer (e.g., at least about two nanometers, at least about five nanometers, at least about 10 nanometers, at least about 25 nanometers, at least about 50 nanometers, at least about 100 nanometers, at least about 250 nanometers, at least about 500 nanometers, at least about one micron, at least about five microns, at least about 10 microns, at least about 25 microns, at least about 50 microns, at least about 75 microns). For example, a silver particle 34 may have a diameter of about 20 nanometers.

A silver particle 34 can be spherical or non-spherical. In some embodiments, a silver particle 34 can be in the form of a flake or a fiber. The fiber can have a circular cross-section or a non-circular (e.g., oval, polygonal) cross-section. In certain embodiments, the fiber can be flat. In some embodiments, the fiber can be in the shape of a ribbon (e.g., a flat or wavy ribbon). Along its length, the fiber can, for example, be straight, wavy, coiled, and/or folded.

As shown in FIG. 1E, generally tubular member 24 has a length L. In some embodiments, length L can be at least about 20 centimeters (e.g., at least about 40 centimeters, at least about 60 centimeters, at least about 80 centimeters, at least about 100 centimeters, at least about 150 centimeters, at least about 200 centimeters) and/or at most about 250 centimeters (e.g., at most about 200 centimeters, at most about 150 centimeters, at most about 100 centimeters, at most about 80 centimeters, at most about 60 centimeters, at most about 40 centimeters). For example, in certain embodiments, length L can be about 60 centimeters. Silver particles 34 can be uniformly distributed in composite 30 along the entire length L of generally tubular member 24, or can be included in one or more selected portions of generally tubular member 24. For example, as FIG. 1E shows, when access catheter 10 is used to deliver therapeutic agents into superior vena cava 37, a first portion E of access catheter 10 is not delivered into body 12 (i.e., first portion E remains on the exterior of body 12), a second portion S/T of access catheter 10 contacts or is disposed within skin and tissue, and a third portion V of access catheter 10 contacts or is disposed within veins. In some embodiments, one or two of these portions can include silver particles 34, while the other portion or portions do not include any silver particles 34. As an example, in certain embodiments, portions S/T and V of generally tubular member 24 can include silver particles 34, while portion E does not include any silver particles 34. The likelihood of infection at the location of portion S/T and/or the location of portion V can be higher than the likelihood of infection at the location of portion E.

In certain embodiments, the presence of silver particles 34 in one or more portions of access catheter 10 may render those portions radiopaque, so that the positions of those portions within body 12 can be determined using X-ray fluoroscopy. For example, as shown in FIG. 1E, access catheter 10 includes a distal portion D at distal end 28 of generally tubular member 24. In some embodiments, it may be desirable for distal portion D to be radiopaque, so that X-ray fluoroscopy can be used to ascertain the position of distal portion D within body 12 (e.g., during delivery of generally tubular member 24). Thus, in certain embodiments, distal portion D may include silver particles 34, while one or more other portions of access catheter 10 (e.g., the remainder of portion V) may not include any silver particles 34.

In some embodiments, a relatively small percent by volume of silver particles 34 can be used in generally tubular member 34, while still providing generally tubular member 34 with radiopacity and/or antimicrobial activity. This can, for example, result in composite 30 exhibiting mechanical and/or chemical properties that are the same as, or similar to, the mechanical and/or chemical properties of polymer 32. In certain embodiments, the presence of a relatively small percent by volume of silver particles 34 in composite 30 can allow generally tubular member 24 to exhibit chemical resistance. The chemical resistance may be similar to the chemical resistance generally tubular member 24 might exhibit if generally tubular member 24 were formed solely of polymer 32. In some embodiments, generally tubular member 24 can exhibit alcohol resistance. This alcohol resistance can, for example, help generally tubular member 24 to retain its shape during use. For example, access catheter 10 may be disposed in a body of a patient for a relatively long period of time (e.g., more than one month). To limit the likelihood of infection, one or more portions generally tubular member 24 may be cleaned (e.g., by the patient) by swabbing the portions with alcohol. Generally tubular member 24 may be relatively unlikely to absorb a significant amount of this alcohol, thereby swelling as a result.

While the percent by volume of silver in generally tubular member 24 and/or composite 30 has been described, in some embodiments, generally tubular member 24 and/or composite 30 can include at least about 0.5 percent by weight silver (e.g., at least about one percent by weight silver, at least about five percent by weight silver, at least about 10 percent by weight silver, at least about 15 percent by weight silver, at least about 20 percent by weight silver, at least about 30 percent by weight silver, at least about 40 percent by weight silver, at least about 50 percent by weight silver, at least about 60 percent by weight silver,) and/or at most about 70 percent by weight silver (e.g., at most about 60 percent by weight silver, at most about 50 percent by weight silver, at most about 40 percent by weight silver, at most about 30 percent by weight silver, at most about 20 percent by weight silver, at most about 15 percent by weight silver, at most about 10 percent by weight silver, at most about five percent by weight silver, at most about one percent by weight silver).

As described above, composite 30 includes a polymer 32. Examples of polymers include thermoplastic polymers (e.g., semi-crystalline thermoplastic polymers) and thermoset polymers. Examples of thermoplastic polymers include polyolefins, polyamides (e.g., polyamide 12, polyamide 11, Nylon, polyamide 6-12), polyesters, polyethers, polyurethanes, polyureas, polyvinyls, polyacrylics, fluoropolymers, copolymers (e.g., block copolymers such as multi-block copolymers), and mixtures thereof. Examples of polyolefins include ethylene vinyl acetate (EVA), high-density polyethylene (HDPE), medium-density polyethylene (MDPE), and low-density polyethylene (LDPE). In some embodiments, a thermoplastic polyurethane can be polyester-, polyether-, polycarbonate-, or polysiloxane-based. Examples of polyurethanes include Tecoflex® polyurethanes (e.g., Tecoflex® 80A), Carbothane® polyurethanes (e.g., Carbothane® 85A polyurethane), and Tecothane® polyurethanes (all from Noveon, Inc., Cleveland, Ohio). An example of a polycarbonate-urethane is Bionate® polycarbonate-urethane (from the Polymer Technology Group, Inc., Berkeley, Calif.). Examples of thermoset polymers include elastomers such as ethylene-propylene terpolymer (EPDM), nitrile butadiene elastomers, silicones, epoxies, ioscyanates, polycaprolactone, and poly(dimethylsiloxane)-containing polyurethanes and ureas. In some embodiments, polymer 32 can be a polyether block amide elastomer (e.g., Pebax® polyether block amide elastomer, available from Arkema Inc., Philadelphia, Pa.). In certain embodiments, polymer 32 can be an amorphous polymer, such as polystyrene, polyvinyl chloride (PVC), acrylonitrile butadiene styrene (ABS), polycarbonate, or polyvinylidene fluoride. In some embodiments, polymer 32 can be an electroactive polymer (EAP). In certain embodiments, polymer 32 can be a piezoelectric polymer (e.g., polyvinylidene fluoride). In some embodiments, polymer 32 can be styrene-butadiene-styrene (SBS) or styrene-isobutylene-styrene (SIBS). In certain embodiments, a composite can include multiple (e.g., two, three, four, five) polymers.

Composite 30 can be formed by any of a number of different methods. For example, composite 30 can be formed by compounding silver particles 34 into polymer 32 using one or more solution dispersion methods, single screw compounding methods, and/or twin screw compounding methods. As an example, in some embodiments, composite 30 can be formed using a dispersion method that includes dissolving polymer 32 in a solvent system to form a solution, adding silver particles 34 into the solution to form a mixture, mixing the mixture, pouring the mixture onto a piece of filter paper to separate the solids in the mixture from the solvent, drying the filter paper under vacuum to remove residual solvent, and flaking the resulting composite 30 off of the filter paper. As another example, in certain embodiments, composite 30 can be formed using a single screw or twin screw compounding method that includes melting polymer 32, dispersing silver particles 34 into the molten polymer 32, and cooling the resulting mixture until it reaches the solid state.

In certain embodiments, generally tubular member 24 of access catheter 10 can have a relatively high tensile strength (e.g., a tensile strength of at least about 3,000 psi, a tensile strength of at least about 6,000 psi), resistance to tear, and/or flexural modulus (e.g., a flexural modulus of at least about 1,000 psi, a flexural modulus of at least about 1,500 psi). The tensile strength of a generally tubular member such as generally tubular member 24 can be measured, for example, using ASTM D638. The flexural modulus of a block of the composite 30 out of which generally tubular member 24 is formed can be measured, for example, using ASTM D790.

As described above with reference to FIG. 1D, generally tubular member 24 has an inner diameter ID and an outer diameter OD. In some embodiments, outer diameter OD can be at least about one French and/or at most about 20 French. In certain embodiments, inner diameter ID can be at least about 0.5 French and/or at most about 19 French. In some embodiments, access catheter 10 can have a size of from two French to nine French (e.g., from five French to nine French).

Generally tubular member 24 can be formed out of composite 30 using any of a number of different methods, such as an extrusion method and/or a molding method.

As described above, access catheter 10 can be used to deliver one or more therapeutic agents (e.g., chemotherapy drugs) to a target site (e.g., superior vena cava 37). Therapeutic agents—are described, for example, in DiMatteo et al., U.S. Patent Application Publication No. US 2004/0076582 A1, published on Apr. 22, 2004, and entitled “Agent Delivery Particle”, in Pinchuk et al., U.S. Pat. No. 6,545,097, in Schwarz et al., U.S. Pat. No. 6,368,658, and in DiCarlo et al., U.S. patent application Ser. No. 11/111,511, filed on Apr. 21, 2005, and entitled “Particles”, all of which are incorporated herein by reference.

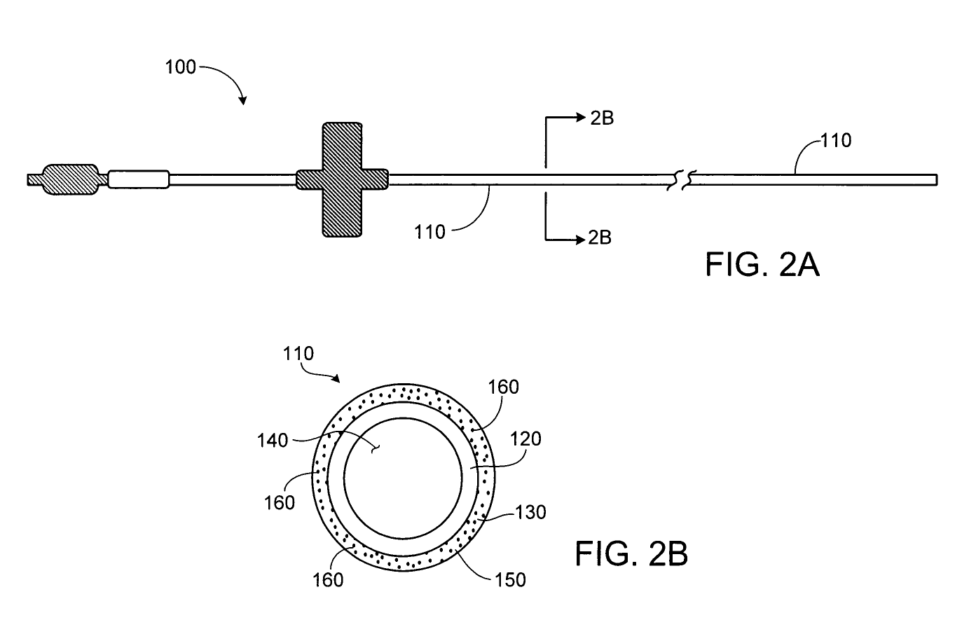

While generally tubular member 24 of access catheter 10 is formed of one layer, in some embodiments, one or more of the components of a catheter can be formed of multiple (e.g., two, three, four, five, 10) layers. For example, FIG. 2A shows an access catheter 100 including a generally tubular member 110. As shown in FIG. 2B, generally tubular member 110, which has a lumen 140, is formed of an inner polymer layer 120 and an outer composite layer 130. Composite layer 130 includes a polymer 150 and silver particles 160. Polymer 150 in composite layer 130 can be the same as, or different from, the polymer in polymer layer 120. Access catheter 100 can be formed, for example, by dissolving polymer 150 in a solvent (e.g., THF, chloroform) to form a solution, adding silver particles 160 into the solution to form a mixture, agitating the mixture to disperse silver particles 160 throughout the mixture, extruding a tube of polymer to form inner polymer layer 120, dipping the tube into the solution, and allowing the solvent to evaporate from the surface of the tube, to form outer composite layer 130. In some embodiments, the thickness of outer composite layer 130 can be increased by repeating the dipping process (e.g., by dipping the tube in the mixture multiple times).

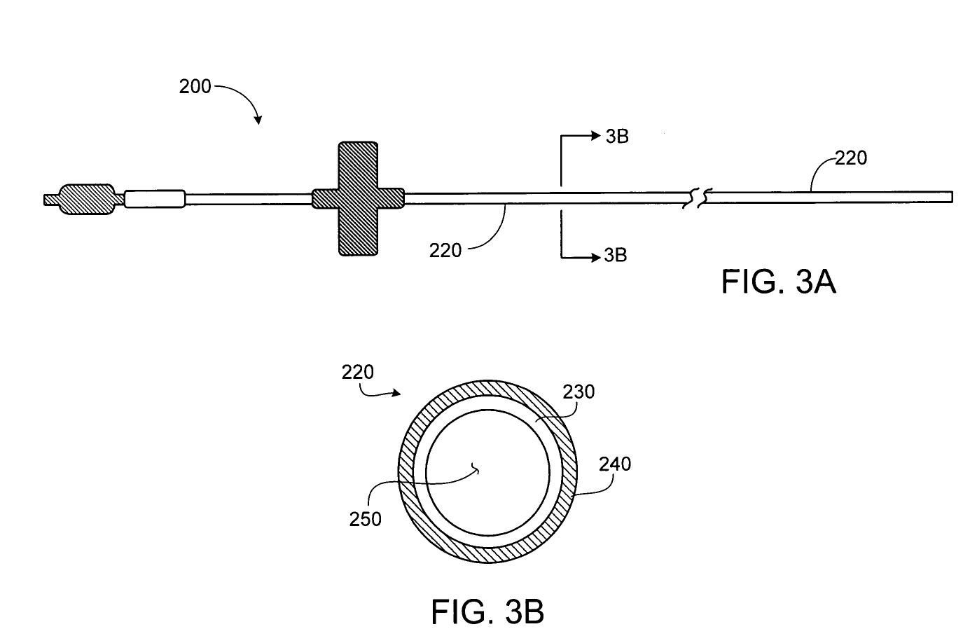

While access catheters including composites including silver particles have been described, in some embodiments, an access catheter can alternatively or additionally include silver in one or more other forms. As an example, FIG. 3A shows an access catheter 200 including a generally tubular member 220. As shown in FIG. 3B, generally tubular member 220, which has a lumen 250, is formed of an inner polymer layer 230 and an outer silver coating 240 (formed of elemental silver).

Silver coating 240 can be formed of, for example, plated silver, or can be formed of silver particles deposited onto polymer layer 230 in the form of a coating. Examples of methods that can be used to deposit silver coating 240 onto polymer layer 230 include vapor deposition methods, thin-film deposition methods, plating methods (e.g., electroplating), plasma-arc deposition methods, spraying methods, and dip-coating methods. Vapor deposition methods can include depositing silver and/or silver complexes from a source to a substrate or target (e.g., polymer layer 230) by dissipating metal ions from the source in a vaporous medium. Examples of vapor deposition methods include chemical vapor deposition methods and physical vapor deposition methods, such as sputtering methods (e.g., vacuum-sputter coating methods) and evaporation methods.

In some embodiments, ion beam assisted deposition, which is a combination of physical vapor deposition (PVD) and ion-beam bombardment, can be used to deposit silver coating 240 onto polymer layer 230. During ion beam assisted deposition, a high power electron beam is used to produce a coating material in vapor form. A medical device and/or medical device component is placed in the presence of the vapor, such that individual coating atoms and/or molecules can condense and stick to the surface of the medical device and/or medical device component. Additionally, highly energetic ions are formed and directed at the surface of the medical device and/or medical device component, resulting in a concurrent ion bombardment that intermixes coating and substrate atoms. As a result, a relatively dense film structure of the coating material can form on the surface of the medical device and/or medical device component. Ion bean assisted deposition is described, for example, in Chandrasekaran et al., U.S. Patent Application Publication No. US 2004/0068315 A1, published on Apr. 8, 2004, and entitled “Medical Devices and Methods of Making the Same”, which is incorporated herein by reference.

Generally tubular member 220 of access catheter 200 may include, for example, the same percent by volume of silver as generally tubular member 24 of access catheter 10. For example, in certain embodiments, generally tubular member 220 can include at least about three percent by volume (e.g., at least about four percent by volume, at least about five percent by volume, at least about six percent by volume, at least about seven percent by volume, at least about eight percent by volume, at least about nine percent by volume, at least about 10 percent by volume, at least about 15 percent by volume, at least about 20 percent by volume, at least about 25 percent by volume, at least about 30 percent by volume, at least about 40 percent by volume, at least about 50 percent by volume), and/or at most about 60 percent by volume (e.g., at most about 50 percent by volume, at most about 40 percent by volume, at most about 30 percent by volume, at most about 25 percent by volume, at most about 20 percent by volume, at most about 15 percent by volume, at most about 10 percent by volume, at most about nine percent by volume, at most about eight percent by volume, at most about seven percent by volume, at most about six percent by volume, at most about five percent by volume, at most about four percent by volume), of silver. In certain embodiments, at least a portion of generally tubular member 220 can include more than five percent by volume (e.g., about 6.4 percent by volume) of silver.

In some embodiments, the percent by volume of silver in generally tubular member 220 can be calculated as follows. First, the mass of silver to be used in forming silver coating 240 is measured using a balance. The mass of the silver is then divided by the density of the silver to provide the volume of the silver. After generally tubular member 220 has been formed using the measured volume of silver, the inner diameter (ID₂₂₀), outer diameter (OD₂₂₀), and length (L₂₂₀) of generally tubular member 220 are measured using, for example, a laser micrometer (from Beta LaserMike, Dayton, Ohio), an optical comparator (from Vision Engineering), and/or scanning electron microscopy (SEM). The volume of generally tubular member 220 is then calculated according to equation (2) below.
volume of member 220=[(π)((0.5)(OD₂₂₀))²−(π)((0.5)(ID₂₂₀))²]×L₂₂₀ (2)

The percent by volume of silver in generally tubular member 220 is then calculated according to equation (3) below:

$$silver percent by volume = \frac{( volume of silver in member 220 )}{( volume of member 220 )} \times 100 \% \qquad ( 3 )$$

Examples of access catheters that can be coated using one or more of the above-described methods include peripherally inserted central catheters (PICC's) and central venous catheters (CVC's). In certain embodiments, a PICC can have a size of from four French to six French. A PICC may be used, for example, for a period of about 30 days or less. In some embodiments, a CVC can have a size of from five French to nine French. A CVC may be used, for example, for a period of about 90 days or more. Examples of commercially available access catheters include the Vaxcel® Peripherally Inserted Central Catheter (PICC) (from Boston Scientific Corp.), and the Vaxcel® PICC With PASV® Valve Technology (from Boston Scientific Corp.).

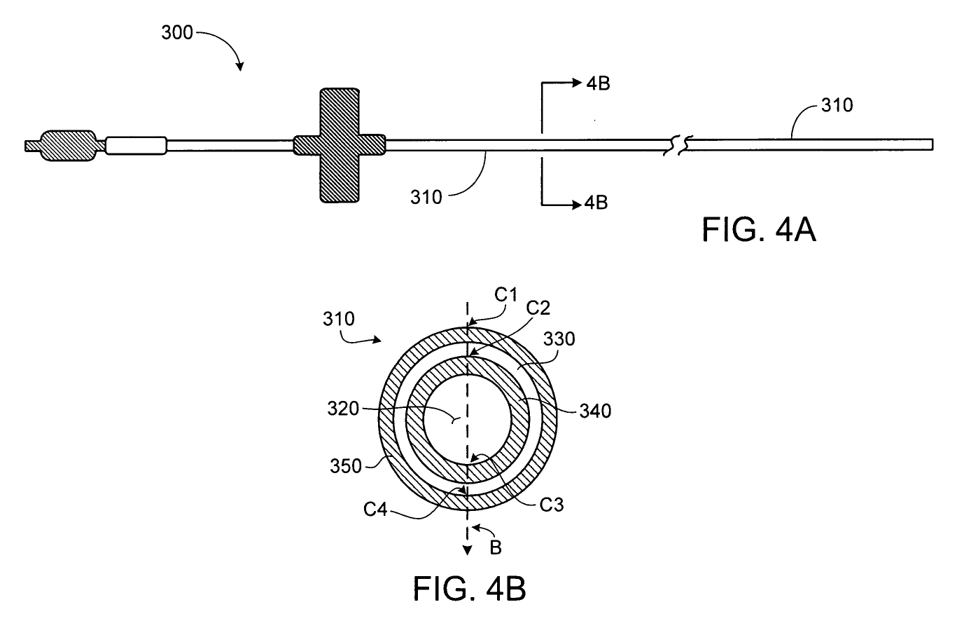

In some embodiments, an access catheter can include a generally tubular member having both an inner silver coating and an outer silver coating. For example, FIG. 4A shows an access catheter 300 including a generally tubular member 310. As shown in FIG. 4B, generally tubular member 310, which has a lumen 320, is formed of an intermediate polymer layer 330 having an inner silver coating 340 and an outer silver coating 350. The presence of silver coatings 340 and 350 can significantly enhance the visibility of generally tubular member 310 under X-ray fluoroscopy. For example, FIG. 4B shows an X-ray beam B traveling through generally tubular member 310. While generally tubular member 310 includes two silver coatings 340 and 350, X-ray beam B contacts silver four different times when traveling through generally tubular member 310: initially at point C1 then at point C2, then at point C3, and finally at point C4.

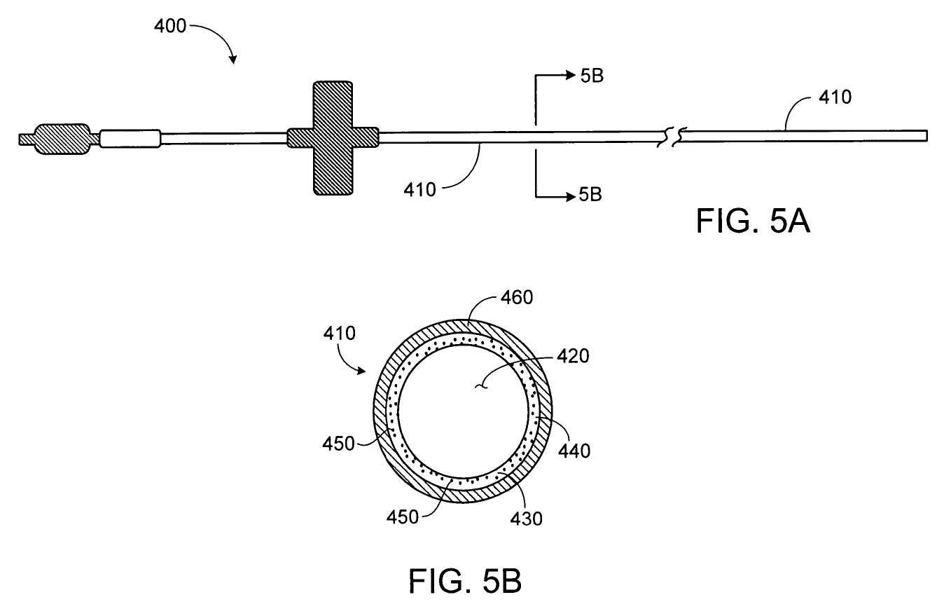

In certain embodiments, an access catheter can include silver in different forms. For example, FIG. 5A shows an access catheter 400 including a generally tubular member 410. As shown in FIG. 5B, generally tubular member 410, which has a lumen 420, is formed of an inner composite layer 430 including a polymer 440 and silver particles 450, and an outer silver coating 460.

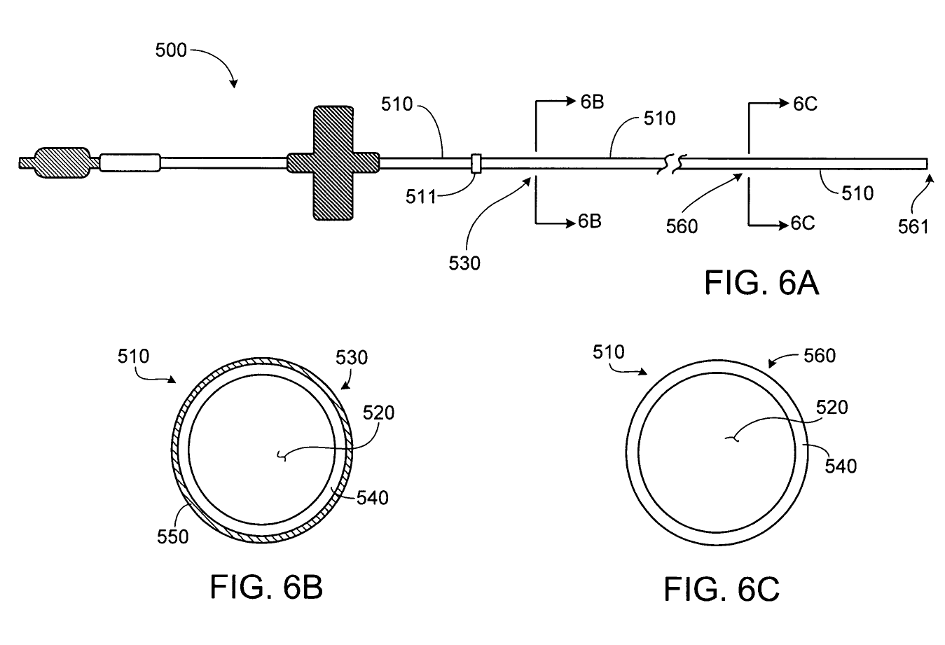

As described above, in some embodiments, an access catheter can include a generally tubular member having one or more portions that include silver, and one or more portions that do not include silver. As an example, FIG. 6A shows an access catheter 500 including a generally tubular member 510 having a lumen 520 (shown in FIGS. 6B and 6C). Access catheter 500 also includes a cuff 511 (e.g., formed of a polyester) on generally tubular member 510. Cuff 511 can, for example, help to secure access catheter 500 in the body, and/or can be located, for example, in a region of generally tubular member 510 that contacts skin and/or tissue during use. As shown in FIGS. 6B and 6C, one portion 530 of generally tubular member 510 is formed of an inner polymer layer 540 and an outer silver coating 550, while another portion 560 of generally tubular member 510 is formed just of polymer layer 540. While two portions of a generally tubular member including the same polymer layer have been described, in certain embodiments, an access catheter can include a generally tubular member having two portions including different polymers. For example, the generally tubular member may have one portion including a layer formed of Carbothane® 75A polyurethane and a silver coating, and another portion including a layer formed of Carbothane® 95A polyurethane. The polymer layers of the different portions may, for example, be formed separately and then butt-welded and/or laminated to each other to form the generally tubular member. While FIGS. 6A-6C show generally tubular member 510 having one portion 530 with a silver coating, in some embodiments, a generally tubular member such as generally tubular member 510 may alternatively or additionally include silver in one or more other portions. For example, the distal end 561 of generally tubular member 530 may include a silver coating.

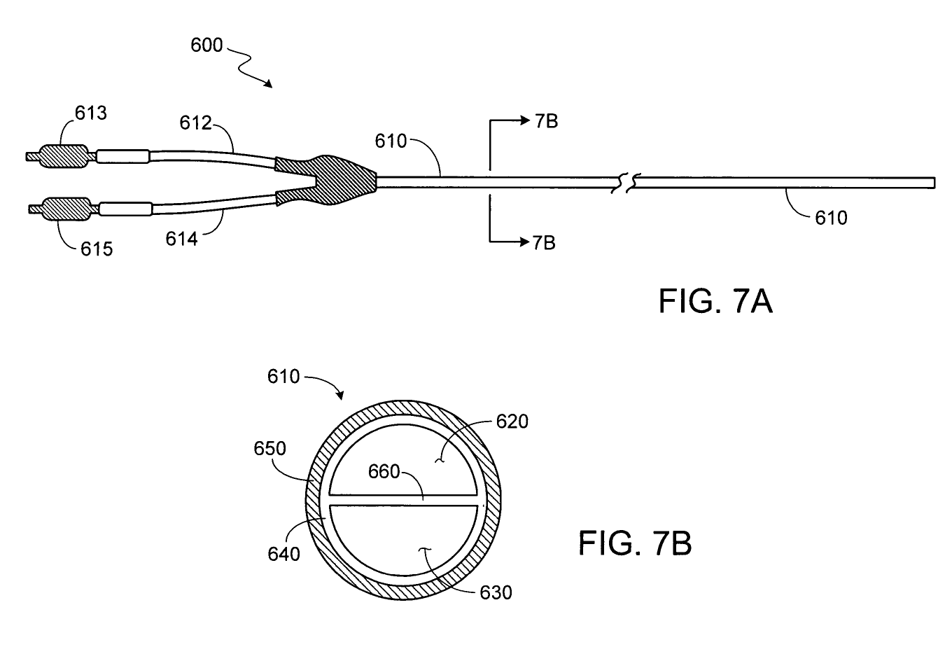

While catheters including generally tubular members having a single lumen have been described, in some embodiments, a catheter can include a member having multiple (e.g., two, three, four, five) lumens. For example, FIG. 7A shows a catheter 600 including an elongated member 610 in fluid communication with two lines 612 and 614. Line 612 is in fluid communication with a valve 613, and line 614 is in fluid communication with a valve 615. As shown in FIG. 7B, elongated member 610 has two lumens 620 and 630. Elongated member 610 is formed of a polymer layer 640 and a silver coating 650. A polymer septum 660 that is integrally formed with polymer layer 640 forms lumens 620 and 630. Catheter 600 can be used, for example, to deliver two different therapeutic agents to a target site simultaneously, without causing the agents to contact each other prior to reaching the target site. For example, line 612 can be in fluid communication with lumen 620 and not with lumen 630, while line 614 can be in fluid communication with lumen 630 and not with lumen 620. A therapeutic agent can be injected into line 612 so that it flows through lumen 620 and into the target site, and another, different, therapeutic agent can be injected into line 614 so that it flows through lumen 630 and into the target site.

While certain embodiments have been described, other embodiments are possible.

As an example, in some embodiments, an access catheter can be connected to a pump. The pump can be used, for example, to pump one or more therapeutic agents through a generally tubular member of the access catheter and into a target site.

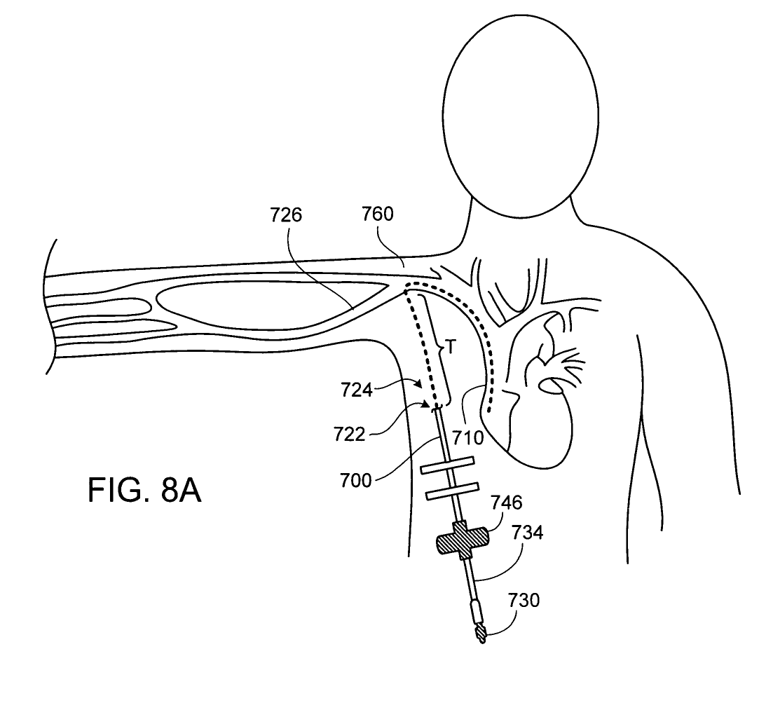

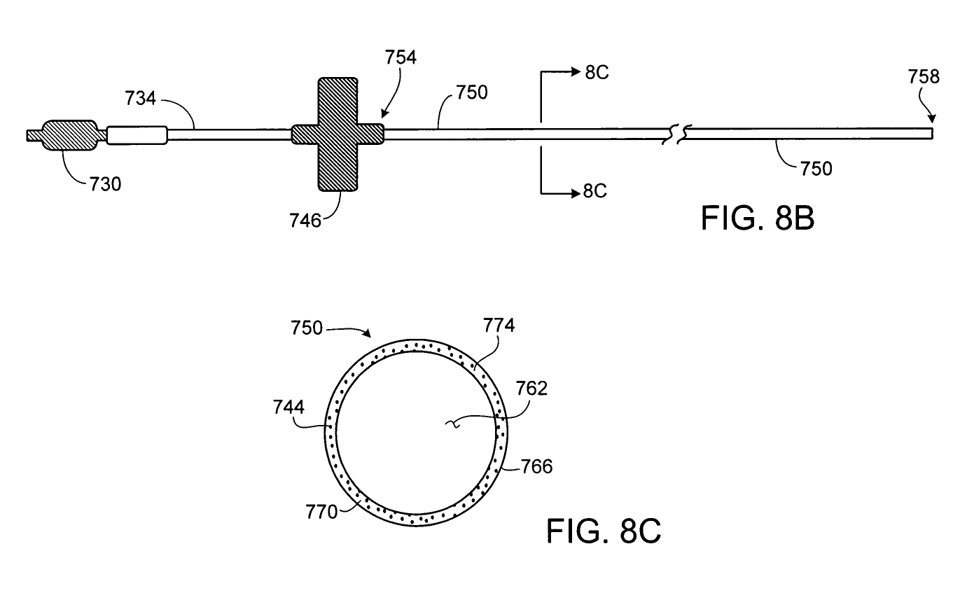

As another example, while access catheters including silver have been described, in some embodiments, a different type of catheter can include silver (e.g., in the volume percents and/or weight percents provided above). For example, FIG. 8A illustrates the delivery of a tunneled catheter 700 into the superior vena cava 710 of a body 720 of a subject. As shown in FIG. 8A, tunneled catheter 700 is delivered into a site 722 in the right chest wall 724 of body 720, and tunnels through a region T of tissue in body 720 before entering axillary vein 726. Once tunneled catheter has been placed within body 720, a portion of tunneled catheter 700 remains tunneled in tissue of body 720, while another portion of tunneled catheter 700 is located within veins of body 720. An example of a commercially available tunneled catheter is the Vaxcel® Tunneled Central Venous Catheter (CVC) (from Boston Scientific Corp.).

Referring now to FIGS. 8B and 8C, tunneled catheter 700 includes a valve 730 that is connected to a line 734, which in turn is connected to a hub 746. Hub 746 is in fluid communication with a generally tubular member 750 having a proximal end 754 and a distal end 758. Generally tubular member 750 has a lumen 762, and is formed of a composite 766 including a polymer 770 and silver particles 774 dispersed within polymer 770.

Other examples of catheters that can include silver include port venous access catheters (VAC's), dialysis catheters (e.g., hemodialysis catheters, such as 14.5 French hemodialysis catheters), and drainage catheters. An example of a commercially available dialysis catheter is the Vaxcel® Plus Chronic Dialysis Catheter (from Boston Scientific Corp.), and examples of commercially available drainage catheters include the Flexima™ Tight Loop All-Purpose Drainage Catheters (from Boston Scientific Corp.). Drainage catheters can be used, for example, for biliary and/or urinary drainage, and/or for draining abscesses and/or collecting fluid. An example of a commercially available drainage set is the Tal MicroDrainage™ Set (from Boston Scientific Corp.).

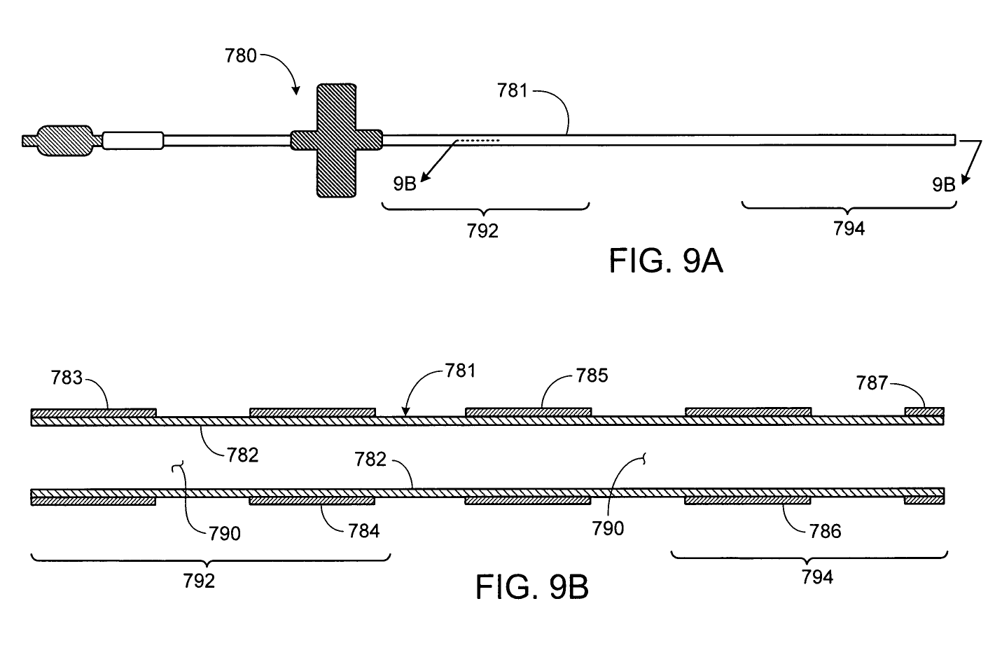

As another example, in some embodiments, an access catheter can include multiple portions including silver, separated from each other by portions that do not include silver. For example, FIG. 9A shows an access catheter 780 including a generally tubular member 781 having a proximal portion 792 and a distal portion 794. When access catheter 780 is used in a subject, proximal portion 792 may be proximate to skin of the subject, and/or distal portion 794 may be disposed within vasculature of the subject. As shown in FIG. 9B, generally tubular member 781, which has a lumen 790, is formed of a polymer layer 782, and is coated in selected regions by silver coatings 783, 784, 785, 786, and 787. The silver coatings may, for example, be separated from each other by a distance of at most about one millimeter.

In some embodiments, silver coatings 783, 784, 785, 786, and/or 787 can be included in regions of generally tubular member 781 that have a higher likelihood of infection than other regions of generally tubular member 781. As an example, a silver coating may be included in a region of generally tubular member 781 that will be located at the interface between skin and air once access catheter 780 has been delivered into the body of a subject. In certain embodiments, silver coatings 783, 784, 785, 786, and/or 787 can be included in regions of generally tubular member 781 that, when viewed using X-ray fluoroscopy, can assist in the navigation and/or placement of generally tubular member 781 at a target site. As an example, silver coatings 786 and 787, which are included in distal portion 794 of generally tubular member 781, may help to make distal portion 794 visible under X-ray fluoroscopy. In some embodiments, silver can leach out from one or more of the silver coatings on generally tubular member 781. This can, for example, result in originally uncoated portions of generally tubular member 781 exhibiting antimicrobial activity and/or radiopacity. In addition, the overall flexibility of generally tubular member 781 is controlled by spacing the coated portions since the mechanical properties of the catheter portions between the coated portions are not substantially affected by the silver. In certain embodiments, generally tubular member 781 can be relatively flexible (e.g., capable of being formed into a ring). While generally tubular member 781 includes multiple silver-coated regions, in some embodiments, a component of an access catheter can include multiple regions including a composite that includes silver. Access catheter 780 can be formed, for example, by selectively coating generally tubular member 781 with silver, and/or by coating generally tubular member 781 with silver along its entire length and then selectively removing portions of the coating (e.g., using a grinding process).

As a further example, in some embodiments, a catheter (e.g., an access catheter) can include silver along the majority of its length, but can also include certain regions in which there is little or no silver. When the catheter is viewed using X-ray fluoroscopy, the regions that do not include silver may be used as markers because they may not be visible under the X-ray fluoroscopy.

As an additional example, while access catheters (e.g., PICC's and CVC's) have been described for therapeutic agent infusion, in some embodiments, an access catheter can be used for other purposes, such as to withdraw blood from a target site (e.g., for testing). Blood can be withdrawn from the target site by, for example, applying suction to one or more valves of the access catheter. In certain embodiments, an access catheter such as a PICC or a CVC may be periodically flushed (e.g., with a saline solution) to reduce the likelihood of blockage formation within the access catheter during use.

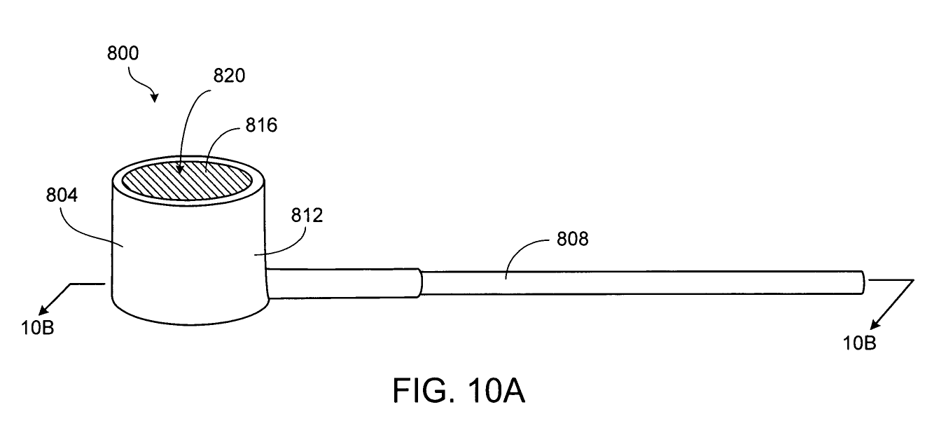

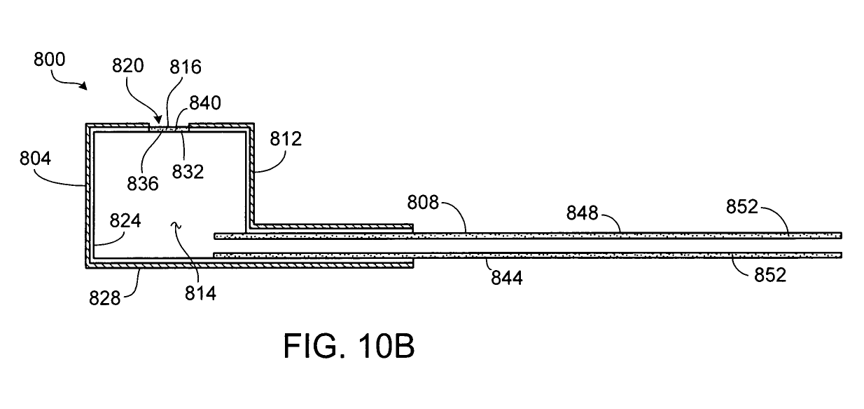

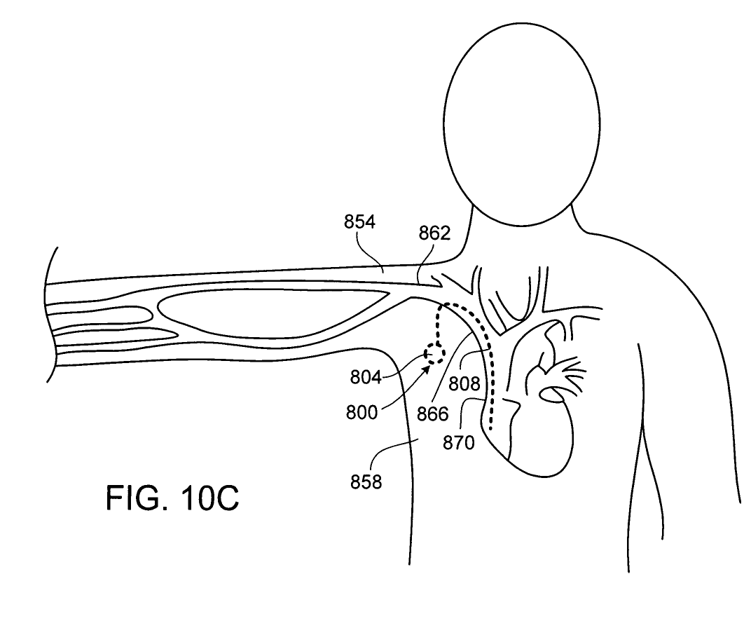

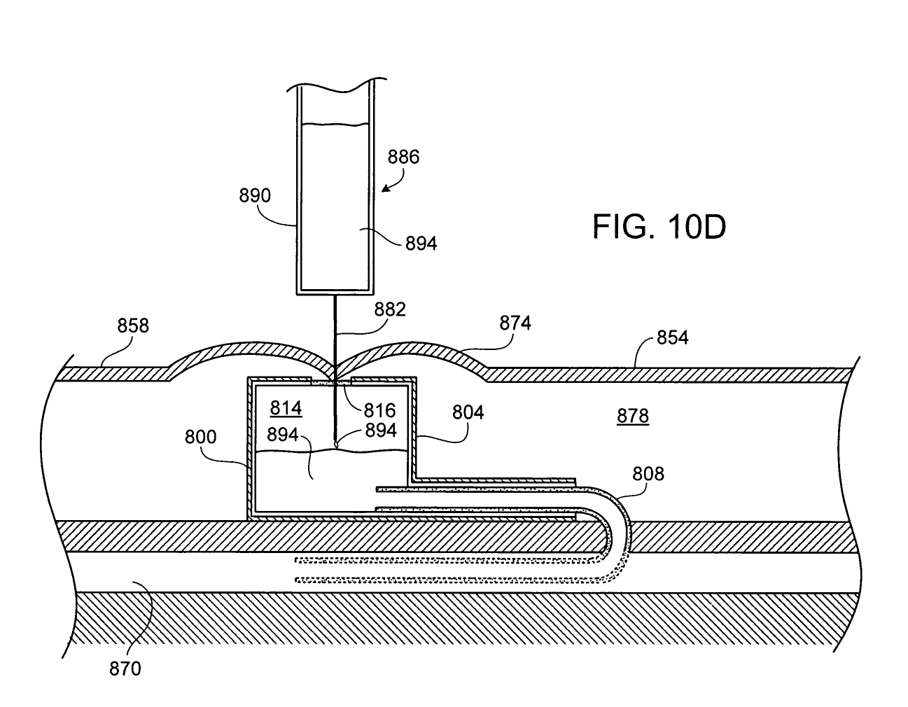

As a further example, while catheters including silver have been described, in some embodiments, one or more other medical devices (e.g., implantable medical devices) can include silver (e.g., in the volume percents and/or weight percents provided above). As an example, in certain embodiments, an implantable port can include silver. For example, FIGS. 10A and 10B show an implantable port system 800 including a port 804 and a catheter 808 in fluid communication with port 804. Port 804 includes a port housing 812 defining a reservoir 814, and a septum 816 on its top surface 820. As shown in FIG. 10B, port housing 812 is formed of a polymer layer 824 and is coated with a silver coating 828. Silver coating 828 does not extend over septum 816. However, septum 816 is formed of a composite 832 including a polymer 836 and silver particles 840. In some embodiments, septum 816 can exhibit antimicrobial activity without exhibiting radiopacity. In certain embodiments, septum 816 can exhibit both antimicrobial activity and radiopacity. Catheter 808 is formed of a composite 844 including a polymer 848 and silver particles 852. Composite 832 of septum 816 and composite 844 of catheter 808 can be the same as, or different from, each other.

FIG. 10C shows port system 800 when it has been implanted into a body 854 of a subject. In some embodiments, port system 800 can be surgically implanted into body 854. As shown in FIG. 10C, port 804 is implanted into the right chest wall 858 of body 854, and catheter 808 is threaded into subclavian vein 862, brachiocephalic vein 866, and superior vena cava 870. Referring also now to FIG. 10D, after port system 800 has been implanted into body 854, port system 800 can be used, for example, to deliver therapeutic agents into superior vena cava 870. As an example, FIG. 10D shows port system 800 disposed within right chest wall 858 of body 854. Port 804 is implanted underneath skin 874, within subcutaneous layer 878. In some embodiments, port 804 can be secured to this location using suturing. The needle 882 of a syringe 886 including a barrel 890 containing a therapeutic agent 894 is injected through septum 816, and therapeutic agent 894 is injected out of barrel 890 and into reservoir 814. The therapeutic agent then flows from reservoir 814, through catheter 808, and into superior vena cava 870. In some embodiments, port system 800 can include one or more valves that can be used to regulate the delivery of therapeutic agent from port 804 into catheter 808 and eventually into the target site.

As another example, in some embodiments, an implantable endoprosthesis, such as a stent, can include silver. As an example, in certain embodiments, one or more portions (e.g., all) of the body of a stent may be formed of a composite including a polymer and silver. As an additional example, in some embodiments, one or more portions (e.g., all) of the body of a stent may be coated with a silver coating. Examples of stents include urethral stents and coronary stents. Stents are described, for example, in Sahatjian et al., U.S. Patent Application Publication No. US 2005/0010275 A1, published on Jan. 13, 2005, and entitled “Implantable Medical Devices”, and in Sahatjian et al., U.S. Patent Application Publication No. US 2005/0216074 A1, published on Sep. 29, 2005, and entitled “Implantable Medical Devices”, both of which are incorporated herein by reference.

As an additional example, in some embodiments, an endoscopy device can include silver. In certain embodiments, an endoscopy device can include one or more polymers, such as a thermoplastic elastomer. Examples of thermoplastic elastomers include Pebax® polyether block amide elastomers (from Arkema Inc., Philadelphia, Pa.). In some embodiments, an endoscopy device can include one or more polyamides. In some embodiments, an endoscopy device can include a composite including silver particles and one or more polymers. The composite may exhibit chemical resistance, such as resistance to bile, which can help the endoscopy device to maintain its structural integrity during use. Examples of endoscopy devices include biliary stents (e.g., the WALLSTENT® RX Biliary Endoprosthesis (from Boston Scientific Corp.)), percutaneous endoscopic gastronomy tubes, and gastrointestinal (GI) stents.

As a further example, in some embodiments, a medical device that includes silver can include at least one other radiopaque material. Examples of radiopaque materials include barium sulfate, bismuth trioxide, gold, platinum, bismuth oxychloride, bismuth subcarbonate, iridium, and tungsten. For example, in certain embodiments, a catheter can include a generally tubular member formed of a composite including a polymer and silver particles and barium sulfate particles dispersed within the polymer.

As an additional example, while certain methods have been described for adding silver to a medical device, in some embodiments, one or more other methods can alternatively or additionally be used. For example, in certain embodiments, silver particles can be implanted and/or impregnated into a medical device (e.g., into a polymer layer of a catheter) using an ion implantation method. An ion implantation method can be conducted in a vacuum chamber at low pressure (e.g., from 10⁻⁵ Torr to 10⁻⁴ Torr). During the ion implantation method, large numbers of ions can be passed through a mass-analyzing magnet that selects desired ions, and then a beam of the selected ions can be accelerated using a potential gradient column. Electrostatic and magnetic lens elements can shape the resulting beam and scan it over an area containing the medical device and/or medical device component to be treated. The ions can then bombard and penetrate the surface of the medical device and/or medical device component (e.g., the surface of a generally tubular member of an access catheter).

As a further example, while medical devices including silver particles formed of elemental silver have been described, in some embodiments, one or more other forms of silver can alternatively or additionally be included in a medical device. As an example, in certain embodiments, one or more silver complexes, such as one or more silver salts, can be included in a medical device. Examples of silver complexes include silver oxide (e.g., argentous oxide (AgO), disilver oxide (Ag₂O)), silver chloride, silver phosphate, silver sulfate, silver nitrate, silver lactate, silver chlorate, silver iodide, silver fluoride, silver bromide, and silver picrate.

The percent by volume of silver in, for example, a generally tubular member formed of a composite formed of a polymer and AgO particles can be determined as follows.

First, prior to forming the composite, the mass of the AgO particles is measured using a balance, and the mass of the polymer is measured (separately) using a balance. The mass of the AgO particles is divided by the density of AgO to provide the volume of the AgO particles. The molar mass of AgO is equal to the sum of the molar mass of silver and the molar mass of oxygen, or 123.8676 grams/mol. Every mole of AgO includes 107.8682 grams of silver and 15.9994 grams of oxygen. The mass of silver (Ag) in one molecular unit of AgO is calculated according to equation (4) below.

$$( mass Ag ) / ( Ag O molecular unit ) = \frac{( 107.8682 grams Ag ) / ( mole of Ag O molecular units )}{( 6.02 \times {10}^{23} Ag O molecular units ) / ( mole of Ag O molecular units )} \qquad ( 4 )$$

The volume of silver (Ag) per molecular unit of AgO is then calculated according to equation (5) below.

$$( volume Ag ) / ( Ag O molecular unit ) = \frac{( mass Ag ) / ( Ag O molecular unit )}{density of Ag} \qquad ( 5 )$$

Next, the mass of one molecular unit of AgO is determined according to equation (6) below.

$$mass of Ag O molecular unit = \frac{( 124 grams of Ag O ) / ( mole AgO molecular units )}{( 6.02 \times {10}^{23} Ag O molecular units ) / ( mole Ag O molecular units )} \qquad ( 6 )$$

The volume of one AgO molecular unit is then calculated according to equation (7) below.
volume of AgO molecular unit=(mass of AgO molecular unit)/(density of AgO) (7)
A volume ratio is then calculated according to equation (8) below.

$$volume ratio = \frac{( volume of Ag per Ag O molecular unit )}{( volume of Ag O molecular unit )} \qquad ( 8 )$$

Next, the volume of silver in the composite is calculated according to equation (9) below:
volume Ag in composite=(equation (8) volume ratio)×(volume AgO in composite) (9)
The volume ratio of silver (Ag) in the composite is then calculated according to equation (10) below.

$$Ag volume ratio = \frac{( volume Ag in composite )}{( volume polymer in composite ) + ( volume Ag O in composite )} \qquad ( 10 )$$

Finally, the percent by volume of silver in the composite (and, therefore, in the generally tubular member) is calculated according to equation (11) below.
percent by volume of silver=(Ag volume ratio from equation (10))×100% (11)

As another example, in some embodiments, silver can be bonded to the surface of a medical device. For example, in certain embodiments, silver wire can be bonded to the surface of a generally tubular member of a catheter using, for example, an adhesive.

As an additional example, in certain embodiments, silver particles can be coated prior to being incorporated into a polymer to form a composite. The silver particles can be coated with, for example, a polymer, such as a thermoset polymer and/or a thermoplastic polymer. In some embodiments, the silver particles can be coated with a solution-grade polymer (e.g., solution-grade polyurethane). The coating can, for example, help to protect the silver particles during the composite formation process. In certain embodiments, the coating can limit the amount of oxidation of the silver particles during the composite formation process. The coating may be applied to the silver particles using, for example, a spraying process. In some embodiments, a polymer coating can be applied to the silver particles by dissolving the polymer in a solvent (e.g., an alcohol solvent), and applying the resulting solution to the particles (e.g., by adding the particles into the solution). The particles can then be dried (e.g., under vacuum). An example of a solution that can be applied to the particles to form a coating is a solution formed of a Tecoflex® resin (from Noveon, Inc.) dissolved in chloroform or dimethylacetamide. Another example of a solution that can be applied to the particles to form a coating is a solution formed of a Tecoflex® 80A resin (from Noveon, Inc.) dissolved in tetrahydrofuran (THF). In some embodiments, the coating can be applied to the particles in an environment having a temperature of about 25° C. and/or having little or no oxygen. For example, in certain embodiments, the coating can be applied to the particles under vacuum, or in an atmosphere including nitrogen gas and not including oxygen.

As another example, in some embodiments, a medical device may have different portions including different volume percents of silver. For example, a portion of a medical device that contacts the skin and/or tissue during use may include silver at a relatively high volume percent, while a different portion of the medical device that contacts the blood during use may include silver at a relatively low volume percent.

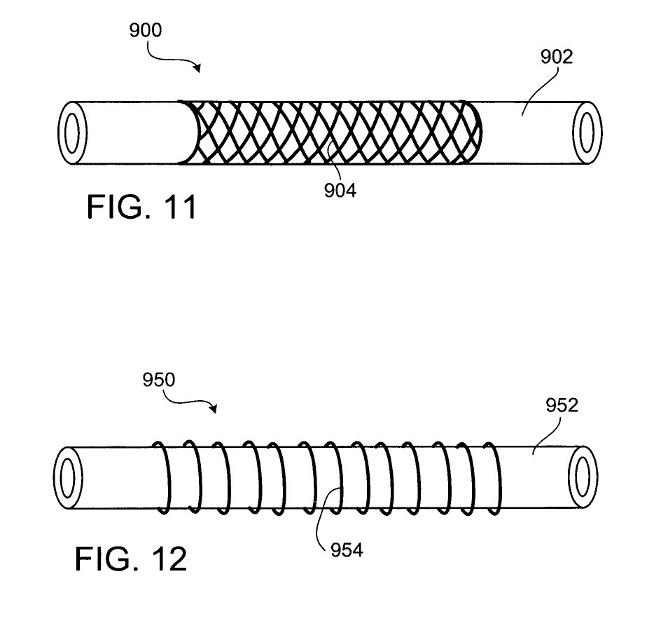

As a further example, in some embodiments, a medical device can include silver mesh. For example, a catheter may include a generally tubular member including a polymer layer and a silver mesh disposed over the polymer layer and/or at least partially embedded (e.g., fully embedded) in the polymer layer. As an example, FIG. 11 shows a generally tubular member 900 of a catheter. Generally tubular member 900 is formed of a polymeric tube 902 that is partially covered by a silver mesh 904. In some embodiments, silver mesh 904 can be adhered to the polymeric tube 902 using an adhesive such as a room-temperature vulcanization (RTV) adhesive. In certain embodiments (e.g., certain embodiments in which polymeric tube 902 is formed of a Tecoflex™ polymer from Noveon, Inc.), one or more alcohols can be used to adhere silver mesh 904 to polymeric tube 902. In some embodiments, a silver mesh can be attached to a polymeric tube using one or more heat-sealable sleeves. As another example, FIG. 12 shows a generally tubular member 950 of a catheter. Generally tubular member 950 is formed of a polymeric tube 952 that is partially covered by a silver coil 954. In certain embodiments, a medical device can include a mesh and/or a coil that is formed of one or more fibers formed of a composite including at least one polymer (e.g., a polyurethane, such as a Pellethane™ thermoplastic polyurethane elastomer from Dow Chemical Co.) and silver particles. The fibers can be formed, for example, by an extrusion process. In some embodiments, silver rings can be attached to a medical device.

As another example, in certain embodiments, a medical device or medical device component can include silver foil. For example, a generally tubular member of a catheter may include a layer formed of silver foil.

As an additional example, while certain methods of delivering medical devices have been described, in some embodiments, other methods can be used. For example, in certain embodiments, a needle can be inserted into a vein and then removed, and a PICC can then be inserted into the location previously occupied by the needle, and can be threaded into the target site.

As a further example, while medical devices including polymers including silver and polymers coated with silver have been described, in some embodiments, a medical device can include one or more other materials that include silver and/or are coated with silver. Examples of other materials include metals (e.g., titanium), metal alloys (e.g., stainless steel), and glass.

As another example, while medical devices including silver-containing composites and/or silver coatings in certain regions of the medical devices have been described, in some embodiments, medical devices can include silver-containing composites and/or silver coatings in other regions. As an example, in certain embodiments, a port can include a housing that is formed of a composite including a polymer and silver particles.

As a further example, while a port with a septum including silver has been described, in some embodiments, a port may include a septum that does not include any silver.

As another example, in some embodiments, a section of a medical device including silver can exhibit both antimicrobial activity and radiopacity, or can exhibit either antimicrobial activity or radiopacity.

As an additional example, while single-lumen access catheters having one valve have been described, in some embodiments, a single-lumen access catheter can have more than one valve (e.g., two valves).

Other embodiments are in the claims.

What is claimed is:

1. An access catheter, comprising:
a generally tubular member insertable through the skin into a body of a subject and including a proximal portion positionable proximate to the skin of the subject and a distal portion, the generally tubular member including a first tubular section comprising a polymer and at least about three percent by volume silver, wherein the first tubular section exhibits radiopacity and antimicrobial activity.

2. The access catheter of claim 1, wherein the first tubular section is selectively located in the proximal portion of the generally tubular member.

3. The access catheter of claim 2, including a second tubular section comprising a polymer and at least about three percent silver, wherein the second tubular section is located in the distal portion of the generally tubular member and is spaced from the first tubular section in the proximal portion of the generally tubular member.

4. The access catheter of claim 1, including a single tubular section located only in the proximal portion of the generally tubular member.

5. The access catheter of claim 1, including a plurality of spaced tubular sections comprising a polymer and at least about three percent by volume silver.

6. The access catheter of claim 1, wherein the silver and the polymer are in the form of a composite.

7. The access catheter of claim 1, wherein the silver is in the form of a coating on the polymer.

8. The access catheter of claim 7, wherein the first tubular section includes an interior surface, and the coating is on the interior surface of the first tubular section.

9. The access catheter of claim 1, wherein the first tubular section includes the silver and the polymer in the form of a composite and the silver in the form of a coating.

10. The access catheter of claim 1, wherein the first tubular section comprises a first layer comprising the polymer.

11. The access catheter of claim 10, wherein the first tubular section further comprises a second layer comprising the silver.

12. The access catheter of claim 11, wherein the second layer is supported by the first layer.

13. The access catheter of claim 11, wherein the first tubular section further comprises a third layer comprising silver.

14. The access catheter of claim 1, wherein the first tubular section comprises at most about 60 percent by volume silver.

15. The access catheter of claim 1, wherein the first tubular section comprises more than five percent by volume silver.

16. The access catheter of claim 1, wherein the silver comprises elemental silver.

17. The access catheter of claim 1, wherein the silver is in the form of a silver complex.

18. The access catheter of claim 1, wherein the silver is in the form of particles.

19. The access catheter of claim 18, wherein the particles have a maximum dimension of at most about 100 microns.

20. The access catheter of claim 18, wherein the particles have a maximum dimension of at most about 100 nanometers.

21. The access catheter of claim 1, wherein the generally tubular member further comprises at least one radiopaque material selected from the group consisting of barium sulfate, bismuth trioxide, gold, platinum, bismuth oxychloride, bismuth subcarbonate, iridium, tungsten, and combinations thereof.

22. A catheter, comprising:
a composite comprising a polymer and at least about three percent by volume silver,
wherein the catheter exhibits radiopacity and antimicrobial activity.

23. The catheter of claim 22, wherein the catheter comprises an access catheter.

24. The catheter of claim 22, further comprising a generally tubular member comprising a first layer comprising the composite.

25. The catheter of claim 24, wherein the generally tubular member further comprises a second layer comprising silver.
